(19) United States
(12) Patent Application Publication
(10) Pub. No.: US 2025/0308057 A1
(43) Pub. Date: Oct. 2, 2025

(54) POSE ESTIMATION USING MACHINE LEARNING

(71) Applicant: Auris Health, Inc., Santa Clara, CA (US)

(72) Inventors: Dingzhong Zhang, Foster City, CA (US); Mohammad Matinfar, San Jose, CA (US)

(73) Assignee: Auris Health, Inc., Santa Clara, CA (US)

(21) Appl. No.: 19/085,919

(22) Filed: Mar. 20, 2025

Related U.S. Application Data
(60) Provisional application No. 63/572,063, filed on Mar. 29, 2024.

(51) Int. Cl. G06T 7/70; G06T 7/12; G06T 17/00; G16H 30/20; G16H 30/40

(52) U.S. Cl. CPC G06T 7/70; G06T 7/12; G06T 17/00; G16H 30/20; G16H 30/40; G06T 2207/20081; G06T 2207/30061

(57) ABSTRACT

This disclosure provides methods, devices, and systems for pose estimation. The present implementations more specifically relate to techniques for determining the pose of a medical instrument within an anatomy. In some aspects, a controller for a medical system may receive image data representing a three-dimensional (3D) model of an anatomy having an instrument disposed therein. The controller generates a point cloud associated with a distal end of the instrument based on the image data and determines a pose of the distal end of the instrument based at least in part on the point cloud and a known geometry of the distal end of the instrument.

### CROSS-REFERENCE TO RELATED APPLICATIONS

This application claims priority and benefit under 35 U.S.C. § 119 (e) to U.S. Provisional Patent Application No. 63/572,063, filed Mar. 29, 2024, which is incorporated herein by reference in its entirety.

### TECHNICAL FIELD

The present disclosure relates to pose estimation of an object, and specifically to pose estimation using machine learning.

### DESCRIPTION OF RELATED ART

During a medical procedure, physicians are presented with various views of a subject's anatomy. The views can include, for example, preoperatively generated model views, three-dimensional (3D) reconstructed model views, computed tomography (CT) image views, endoscopic image views, or the like. As part of the medical procedure, physicians may need to accurately identify position and orientation of a depicted object.

### SUMMARY

This Summary is provided to introduce in a simplified form a selection of concepts that are further described below in the Detailed Description. This Summary is not intended to identify key features or essential features of the claimed subject matter, nor is it intended to limit the scope of the claimed subject matter.

One innovative aspect of the subject matter of this disclosure can be implemented in a controller for a medical system, including a processing system and a memory. The memory stores instructions that, when executed by the processing system, cause the controller to receive image data representing a three-dimensional (3D) model of an anatomy having an instrument disposed therein; generate a point cloud associated with a distal end of the instrument based on the image data; and determine a pose of the distal end of the instrument based at least in part on the point cloud and a known geometry of the distal end of the instrument.

Another innovative aspect of the subject matter of this disclosure can be implemented in a method of pose estimation. The method includes steps of receiving image data representing a 3D model of an anatomy having an instrument disposed therein; generating a point cloud associated with a distal end of the instrument based on the image data; and determining a pose of the distal end of the instrument based at least in part on the point cloud and a known geometry of the distal end of the instrument.

### BRIEF DESCRIPTION OF THE DRAWINGS

Various embodiments are depicted in the accompanying drawings for illustrative purposes and should in no way be interpreted as limiting the scope of the inventions. In addition, various features of different disclosed embodiments can be combined to form additional embodiments, which are part of this disclosure. Throughout the drawings, reference numbers may be reused to indicate correspondence between reference elements.

FIG. 1 illustrates an example medical system, in accordance with one or more examples.

FIG. 2 illustrates a schematic view of different components of the medical system of FIG. 1, in accordance with one or more embodiments.

FIG. 3 illustrates a block diagram depicting various positioning and/or imaging systems/modalities, in accordance with one or more examples.

FIG. 4 illustrates an example block diagram of a tip pose estimation pipeline, in accordance with one or more embodiments.

FIGS. 5A and 5B illustrate example images associated with a segmentation mask generation process, in accordance with one or more embodiments.

FIG. 6 illustrates an example point cloud, in accordance with one or more embodiments.

FIGS. 7A and 7B illustrate example renderings associated with a scope tip heading determination process, in accordance with one or more embodiments.

FIGS. 8A and 8B illustrates example renderings associated with a scope tip alignment process, in accordance with one or more embodiments.

FIG. 9 illustrates an example rendering associated with a scope tip position determination process, in accordance with one or more embodiments.

FIG. 10 illustrates a flow diagram illustrating an instrument pose estimation process, in accordance with one or more embodiments.

FIG. 11 shows a block diagram of an example controller for a medical system, according to some implementations.

FIG. 12 shows an illustrative flowchart depicting an example pose estimation operation, according to some implementations.

### DETAILED DESCRIPTION

The headings provided herein are for convenience only and do not necessarily affect the scope or meaning of the claimed invention. Although certain preferred embodiments and examples are disclosed below, inventive subject matter extends beyond the specifically disclosed embodiments to other alternative embodiments and/or uses and to modifications and equivalents thereof. Thus, the scope of the claims that may arise herefrom is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order dependent. Additionally, the structures, systems, and/or devices described herein may be embodied as integrated components or as separate components. For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

Although certain spatially relative terms, such as “outer,” “inner,” “upper,” “lower,” “below,” “above,” “vertical,” “horizontal,” “top,” “bottom,” “lateral,” “upwardly,” “side,” and similar terms, are used herein to describe a spatial relationship of one device/element or anatomical structure to another device/element or anatomical structure, it is understood that these terms are used herein for ease of description to describe the positional relationship between element(s)/structures(s), such as with respect to the illustrated orientations of the drawings. It should be understood that spatially relative terms are intended to encompass different orientations of the element(s)/structures(s), in use or operation, in addition to the orientations depicted in the drawings. For example, an element/structure described as “above” another element/structure may represent a position that is below or beside such other element/structure with respect to alternate orientations of a subject or element/structure, and vice-versa. It should be understood that spatially relative terms, including those listed above, may be understood relative to a respective illustrated orientation of a referenced figure.

Certain reference numbers are re-used across different figures of the figure set of the present disclosure as a matter of convenience for devices, components, systems, features, and/or modules having features that may be similar in one or more respects. However, with respect to any of the embodiments disclosed herein, re-use of common reference numbers in the drawings does not necessarily indicate that such features, devices, components, or modules are identical or similar. Rather, one having ordinary skill in the art may be informed by context with respect to the degree to which usage of common reference numbers can imply similarity between referenced subject matter. Use of a particular reference number in the context of the description of a particular figure can be understood to relate to the identified device, component, aspect, feature, module, or system in that particular figure, and not necessarily to any devices, components, aspects, features, modules, or systems identified by the same reference number in another figure. Furthermore, aspects of separate figures identified with common reference numbers can be interpreted to share characteristics or to be entirely independent of one another. In some contexts, features associated with separate figures that are identified by common reference numbers are not related and/or similar with respect to at least certain aspects.

### Overview

The present disclosure provides systems, devices, and methods for estimating a pose (e.g., position and/or orientation) of a tip of a medical instrument having a flexible elongated body inside of a subject's anatomy from images captured external to the subject. As an example, the tip may be a distal end of an endoscope.

Existing tip pose estimation techniques may not adequately address inaccuracies caused by variations in shape and curvature of the flexible elongated body, geometry of the tip, various imaging artifacts, presence of a medical tool in the working channel of the body, or the like. Furthermore, those techniques often include one or more manual steps during which mistakes could be introduced.

The present disclosure discloses an automated process using machine leaning to address the above shortcoming by estimating tip pose in a three-dimensional (3D) space generated from the externally captured images. The images may be computed tomography (CT) images, including Cone-Beam Computed Tomography (CBCT) images. Such CT images can provide third-person views of the tip pose in contrast to first-person views captured from endoscopic imaging devices inside of the patient. The estimated tip pose can provide another reference point when aligning the tip with a target during a medical procedure. For example, an estimated tip pose based on CT images can aid in aligning a biopsy needle to a nodule during robotically assisted bronchoscopy.

### Example Medical System

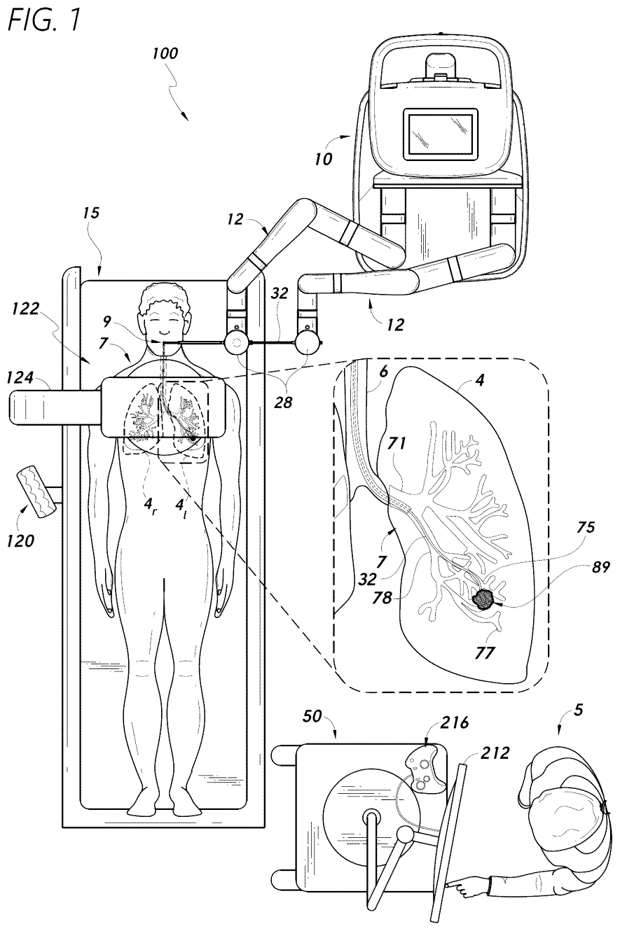

FIG. 1 illustrates an example medical system 100 (also referred to as “surgical medical system 100” or “robotic medical system 100”) in accordance with one or more examples. For example, the medical system 100 can be arranged for diagnostic and/or therapeutic bronchoscopy, as shown. The medical system 100 can include and utilize a robotic system 10, which can be implemented as a robotic cart, for example. Although the medical system 100 is shown as including various cart-based systems/devices, the concepts disclosed herein can be implemented in any type of robotic system/arrangement, such as robotic systems employing rail-based components, table-based robotic end-effectors/manipulators, etc. The robotic system 10 can comprise one or more robotic arms 12 (also referred to as “robotic positioner(s)”) configured to position or otherwise manipulate a medical instrument, such as a medical instrument 32 (e.g., a steerable endoscope or another elongate instrument having a flexible elongated body). For example, the medical instrument 32 can be advanced through a natural orifice access point (e.g., the mouth 9 of a subject 7, positioned on a table 15 in the present example) to deliver diagnostic and/or therapeutic treatment. Although described in the context of a bronchoscopy procedure, the medical system 100 can be implemented for other types of procedures, such as gastro-intestinal (GI) procedures, renal/urological/nephrological procedures, etc. The term “subject” is used herein to refer to live patient as well as any subjects to which the present disclosure may be applicable. For example, the “subject” may refer to subjects including physical anatomic models (e.g., anatomical education model, anatomical model, medical education anatomy model, etc.) used in dry runs, models in computer simulations, or the like that covers non-live patients or test subjects.

With the robotic system 10 properly positioned, the medical instrument 32 can be inserted into the subject 7 robotically, manually, or a combination thereof. In examples, the one or more robotic arms 12 and/or instrument driver(s) 28 thereof can control the medical instrument 32. The instrument driver(s) 28 can be repositionable in space by manipulating the one or more robotic arms 12 into different angles and/or positions.

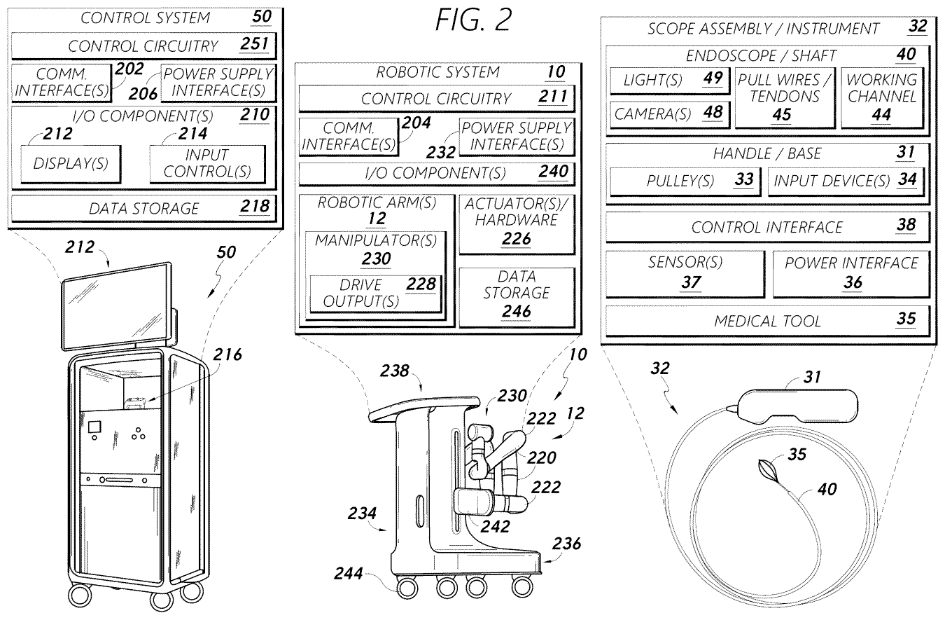

The medical system 100 can also include a control system 50 (also referred to as “control tower” or “mobile tower”), described in detail below with respect to FIG. 2. The control system 50 can include one or more displays 212 to provide/display/present various information related to medical procedures, such as anatomical images. The control system 50 can additionally include one or more control mechanisms, which may be a separate directional input control 216 or a graphical user interface (GUI) presented on the displays 212.

In some embodiments, the display 212 can be a touch-capable display, as shown, that may present anatomical images and allow selection thereon. Few example anatomical images can include CT images, fluoroscopic images, images of an anatomical map, or the like. With the touch-capable display, an operator 5 reviewing the images may find it convenient to identify targets (e.g., target objects or a target region of interest) within the images using a touch-based selection instead of using the directional input control 216. For example, the operator 5 may select a scope tip and/or a nodule using a touchscreen.

The control system 50 can be communicatively coupled (e.g., via wired and/or wireless connection(s)) to the robotic system 10 to provide support for controls, electronics, fluidics, optics, sensors, and/or power to the robotic system 10. Placing such functionality in the control system 50 can allow for a smaller form factor of the robotic system 10 that may be more easily adjusted and/or re-positioned by an operator 5. Additionally, the division of functionality between the robotic system 10 and the control system 50 can reduce operating room clutter and/or facilitate efficient clinical workflow.

The medical system 100 can include an electromagnetic (EM) field generator 120, which is configured to broadcast/emit an EM field that is detected by EM sensors, such as a sensor associated with the medical instrument 32. The EM field can induce small currents in coils of EM sensors (also referred to as “position sensors”), which can be analyzed to determine a pose (position and/or angle/orientation) of the EM sensors relative to the EM field generator 120. In some embodiments, the EM sensors may be positioned at a distal end of the medical instrument 32 and a pose of the distal end may be determined in connection with the pose of the EM sensors. Although EM fields and EM sensors are described in many examples herein, position sensing systems and/or sensors can be any type of position sensing systems and/or sensors, such as optical position sensing systems/sensors, image-based position sensing systems/sensors, etc.

The medical system 100 can further include an imaging system 122 (e.g., a fluoroscopic imaging system) configured to generate and/or provide/send image data (also referred to as “image(s)”) to another device/system. For example, the imaging system 122 can generate image data depicting anatomy of the subject 7 and provide the image data to the control system 50, robotic system 10, a network server, a cloud server, and/or another device. The imaging system 122 can comprise an emitter/energy source (e.g., X-ray source, ultrasound source, or the like) and/or detector (e.g., X-ray detector, ultrasound detector, or the like) integrated into a supporting structure (e.g., mounted on a C-shaped arm support 124), which may provide flexibility in positioning around the subject 7 to capture images from various angles without moving the subject 7. Use of the imaging system 122 can provide visualization of internal structures/anatomy, which can be used for a variety of purposes, such as navigation of the medical instrument 32 (e.g., providing images of internal anatomy to the operator 5), localization of the medical instrument 32 (e.g., based on an analysis of image data), etc. In examples, use of the imaging system 122 can enhance the efficacy and/or safety of a medical procedure, such as a bronchoscopy, by providing clear, continuous visual feedback to the operator 5.

In some examples, the imaging system 122 is a mobile device configured to move around within an environment. For instance, the imaging system 122 can be positioned next to the subject 7 (as illustrated) during a particular phase of a procedure and removed when the imaging system 122 is no longer needed. In other examples, the imaging system 122 can be part of the table 15 or other equipment in an operating environment. The imaging system(s) 122 can be implemented as a Computed Tomography (CT) machine/system, X-ray machine/system, fluoroscopy machine/system, Positron Emission Tomography (PET) machine/system, PET-CT machine/system, CT angiography machine/system, Cone-Beam CT (CBCT) machine/system, 3DRA machine/system, single-photon emission computed tomography (SPECT) machine/system, Magnetic Resonance Imaging (MRI) machine/system, Optical Coherence Tomography (OCT) machine/system, ultrasound machine/system, etc. In some cases, the medical system 100 includes multiple imaging system, such as a first type of imaging system and a second type of imaging system, wherein the different types of imaging systems can be used or positioned over the subject 7 during different phases/portions of a procedure depending on the needs at that time.

In some embodiments, the imaging system 122 can be configured to generate a three-dimensional (3D) model of an anatomy. For example, the imaging system 122 is configured to process multiple images (also referred to as “image data,” in some cases) to generate the 3D model. For example, the imaging system 122 can be implemented as a CT machine configured to capture/generate a series of images/image data (e.g., 2D images/slices) from different angles around the subject 7, and then use one or more algorithms to reconstruct these images/image data into a 3D model. The 3D model can be provided to the control system 50, robotic system 10, a network server, a cloud server, and/or another device, such as for processing, display, or otherwise.

In the interest of facilitating descriptions of the present disclosure, FIG. 1 illustrates a respiratory system as an example anatomy. The respiratory system includes the upper respiratory tract, which comprises the nose/nasal cavity, the pharynx (i.e., throat), and the larynx (i.e., voice box). The respiratory system further includes the lower respiratory tract, which comprises the trachea 6, the lungs 4 (4, and 41), and the various segments of the bronchial tree. The bronchial tree includes primary bronchi 71, which branch off into smaller secondary 78 and tertiary 75 bronchi, and terminate in even smaller tubes called bronchioles 77. Each bronchiole tube is coupled to a cluster of aveoli (not shown). During the inspiration phase of the respiratory cycle, air enters through the mouth and nose and travel down the throat into the trachea 6, into the lungs 4 through the right and left main bronchi 71, into the smaller bronchi airways 78, 75, into the smaller bronchiole tubes 77, and into the alveoli, where oxygen and carbon dioxide exchange takes place.

The bronchial tree is an example luminal network in which robotically-controlled instruments may be navigated and utilized in accordance with the inventive solutions presented here. However, although aspects of the present disclosure are presented in the context of luminal networks including a bronchial network of airways (e.g., lumens, branches) of a subject's lung, some embodiments of the present disclosure can be implemented in other types of luminal networks, such as renal networks, cardiovascular networks (e.g., arteries and veins), gastrointestinal tracts, urinary tracts, etc.

In some embodiments, the imaging system 122 can be configured to capture/update/present images of the anatomy intraoperatively using a CBCT imaging system. During CBCT imaging, the subject 7 may be positioned on the table 15 between an X-ray source and detector mounted on the C-shaped arm support 124 where X-ray beams are passed through a target anatomy, and the resulting images are updated intraoperatively. For example, regarding the lungs 4 of the subject, one or more CBCT captured images or a reconstructed 3D model may be presented to the operator 5 on the display 56. While CBCT is described, it will be understood that the present disclosure contemplates any other imaging techniques capable of providing a 3D reconstruction, such as the normal CT imaging technique.

FIG. 2 illustrates example components of the control system 50, robotic system 10, and medical instrument 32, in accordance with one or more examples. The control system 50 can be coupled to the robotic system 10 and operate in cooperation therewith to perform a medical procedure. For example, the control system 50 can include communication interface(s) 202 for communicating with communication interface(s) 204 of the robotic system 10 via a wireless or wired connection (e.g., to control the robotic system 10). Further, in examples, the control system 50 can communicate with the robotic system 10 to receive position/sensor data therefrom relating to the position of sensors associated with an instrument/member controlled by the robotic system 10. In some examples, the control system 50 can communicate with the EM field generator 120 to control generation of an EM field in an area around a subject 7. The control system 50 can further include a power supply interface(s) 206.

The control system 50 can include control circuitry 251 configured to cause one or more components of the medical system 100 to actuate and/or otherwise control any of the various system components, such as carriages, mounts, arms/positioners, medical instruments, imaging devices, position sensing devices, sensor, etc. Further, the control circuitry 251 can be configured to perform other functions, such as cause display of information, process data, receive input, communicate with other components/devices, and/or any other function/operation discussed herein.

The control system 50 can further include one or more input/out (I/O) components 210 configured to assist a physician or others in performing a medical procedure. For example, the one or more I/O components 210 can be configured to receive input and/or provide output to enable a user to control/navigate the medical instrument 32, the robotic system 10, and/or other instruments/devices associated with the medical system 100. The control system 50 can include one or more displays 212 to provide/display/present various information regarding a procedure. For example, the one or more displays 212 can be used to present navigation information including a virtual anatomical model of anatomy with a virtual representation of a medical instrument, image data, and/or other information.

The one or more I/O components 210 can include a user input control(s) 214, which can include any type of user input (and/or output) devices or device interfaces, such as a directional input control(s) 216, touch-based input control(s) including gesture-based input control(s), motion-based input control(s), or the like. The user input control(s) 214 may include one or more buttons, keys, joysticks, handheld controllers (e.g., video-game-type controllers), computer mice, trackpads, trackballs, control pads, sensors (e.g., motion sensors or cameras) that capture hand gestures and finger gestures, touchscreens, toggle (e.g., button) inputs, and/or interfaces/connectors therefore. In examples, such input(s) can be used to generate commands for controlling medical instrument(s), robotic arm(s), and/or other components.

The control system 50 can also include data storage 218 configured to store executable instruments (e.g., computer-executable instructions) that are executable by the control circuitry 251 to cause the control circuitry 251 to perform various operations/functionality discussed herein. In examples, two or more of the components of the control system 50 can be electrically and/or communicatively coupled to each other.

The robotic system 10 can include the one or more robotic arms 12 configured to engage with and/or control, for example, the medical instrument 32 and/or other elements/components to perform one or more aspects of a procedure. As shown, each robotic arm 12 can include multiple segments 220 coupled to joints 222, which can provide multiple degrees of movement/freedom. The robotic system 10 can be configured to receive control signals from the control system 50 to perform certain operations, such as to position one or more of the robotic arms 12 in a particular manner, manipulate an instrument, and so on. In response, the robotic system 10 can control, using control circuitry 211 thereof, actuators 226 and/or other components of the robotic system 10 to perform the operations. For example, the control circuitry 211 can control insertion/retraction, articulation, roll, etc. of a shaft of the medical instrument 32 or another instrument by actuating a drive output(s) 228 of a manipulator(s) 230 (e.g., end-effectors) coupled to a base of a robotically-controllable instrument. The drive output(s) 228 can be coupled to a drive input on an associated instrument, such as an instrument base of an instrument that is coupled to the associated robotic arm 12. The robotic system 10 can include one or more power supply interfaces 232.

The robotic system 10 can include a support column 234, a base 236, and/or a console 238. The console 238 can provide one or more I/O components 240, such as a user interface for receiving user input and/or a display screen (or a dual-purpose device, such as a touchscreen) to provide the physician/user with preoperative and/or intraoperative data. The support column 234 can include an arm support 242 (also referred to as “carriage”) for supporting the deployment of the one or more robotic arms 12. The arm support 242 can be configured to vertically translate along the support column 234. Vertical translation of the arm support 242 allows the robotic system 10 to adjust the reach of the robotic arms 12 to meet a variety of table heights, subject sizes, and/or physician preferences. The base 236 can include wheel-shaped casters 244 (also referred to as “wheels 244”) that allow for the robotic system 10 to move around the operating room prior to a procedure. After reaching the appropriate position, the casters 244 can be immobilized using wheel locks to hold the robotic system 10 in place during the procedure.

The joints 222 of each robotic arm 12 can each be independently-controllable and/or provide an independent degree of freedom available for instrument navigation. In some examples, each robotic arm 12 has seven joints, and thus provides seven degrees of freedom, including “redundant” degrees of freedom. Redundant degrees of freedom can allow robotic arms 12 to be controlled to position their respective manipulators 230 at a specific position, orientation, and/or trajectory in space using different linkage positions and joint angles. This allows for the robotic system 10 to position and/or direct a medical instrument from a desired point in space while allowing the physician to move the joints 222 into a clinically advantageous position away from the patient to create greater access, while avoiding collisions.

The one or more manipulators 230 (e.g., end-effectors) can be couplable to an instrument base/handle, which can be attached using a sterile adapter component in some instances. The combination of the manipulator 230 and coupled instrument base, as well as any intervening mechanics or couplings (e.g., sterile adapter), can be referred to as a manipulator assembly, or simply a manipulator. Manipulator/manipulator assemblies can provide power and/or control interfaces. For example, interfaces can include connectors to transfer pneumatic pressure, electrical power, electrical signals, and/or optical signals from the robotic arm 12 to a coupled instrument base. Manipulator/manipulator assemblies can be configured to manipulate medical instruments (e.g., surgical tools/instruments) using techniques including, for example, direct drives, harmonic drives, geared drives, belts and/or pulleys, magnetic drives, and the like.

The robotic system 10 can also include data storage 246 configured to store executable instruments (e.g., computer-executable instructions) that are executable by the control circuitry 211 to cause the control circuitry 211 to perform various operations/functionality discussed herein. In example, two or more of the components of the robotic system 10 can be electrically and/or communicatively coupled to each other.

Data storage (including the data storage 218, data storage 246, and/or other data storage/memory) can include any suitable or desirable type of computer-readable media. For example, computer-readable media can include one or more volatile data storage devices, non-volatile data storage devices, removable data storage devices, and/or nonremovable data storage devices implemented using any technology, layout, and/or data structure(s)/protocol, including any suitable or desirable computer-readable instructions, data structures, program modules, or other types of data.

Computer-readable media that can include, but is not limited to, phase change memory, static random-access memory (SRAM), dynamic random-access memory (DRAM), other types of random access memory (RAM), read-only memory (ROM), electrically erasable programmable read-only memory (EEPROM), flash memory or other memory technology, compact disk read-only memory (CD-ROM), digital versatile disks (DVD) or other optical storage, magnetic cassettes, magnetic tape, magnetic disk storage or other magnetic storage devices, or any other non-transitory medium that can be used to store information for access by a computing device. As used in certain contexts herein, computer-readable media may not generally include communication media, such as modulated data signals and carrier waves. As such, computer-readable media should generally be understood to refer to non-transitory media.

Control circuitry (including the control circuitry 251, control circuitry 211, and/or other control circuitry) can include circuitry embodied in a robotic system, control system/tower, instrument, or any other component/device. Control circuitry can include any collection of processors, processing circuitry, processing modules/units, chips, dies (e.g., semiconductor dies including one or more active and/or passive devices and/or connectivity circuitry), microprocessors, micro-controllers, digital signal processors, microcomputers, central processing units, field-programmable gate arrays, programmable logic devices, state machines (e.g., hardware state machines), logic circuitry, analog circuitry, digital circuitry, and/or any device that manipulates signals (analog and/or digital) based on hard coding of the circuitry and/or operational instructions. Control circuitry referenced herein can further include one or more circuit substrates (e.g., printed circuit boards), conductive traces and vias, and/or mounting pads, connectors, and/or components. Control circuitry can further comprise one or more storage devices, which may be embodied in a single device, a plurality of devices, and/or embedded circuitry of a device. Such data storage can comprise read-only memory, random access memory, volatile memory, non-volatile memory, static memory, dynamic memory, flash memory, cache memory, data storage registers, and/or any device that stores digital information. In examples in which control circuitry comprises a hardware and/or software state machine, analog circuitry, digital circuitry, and/or logic circuitry, data storage device(s)/register(s) storing any associated operational instructions can be embedded within, or external to, the circuitry comprising the state machine, analog circuitry, digital circuitry, and/or logic circuitry.

Functionality described herein can be implemented by the control circuitry 251 of the control system 50 and/or the control circuitry 211 of the robotic system 10, such as by the control circuitry 251, 211 executing executable instructions to cause the control circuitry 251, 211 to perform the functionality.

The scope assembly/medical instrument 32 includes a handle or base 31 coupled to an endoscope shaft. For example, an endoscope 40 (also referred herein as “scope” or “shaft”) can include the elongate shaft including one or more lights 49 and one or more cameras 48 or other imaging devices. The medical instrument 32 can be powered through a power interface 36 and/or controlled through a control interface 38, each or both of which may interface with a robotic arm/component of the robotic system 10. The medical instrument 32 may further comprise one or more sensors 37, such as pressure sensors and/or other force-reading sensors, which may be configured to generate signals indicating forces experienced at/by one or more components of the medical instrument 32.

The medical instrument 32 includes certain mechanisms for causing the scope 40 to articulate/deflect with respect to an axis thereof. For example, the scope 40 may have been associated with a proximal portion thereof, one or more drive inputs 34 associated, and/or integrated with one or more pulleys/spools 33 that are configured to tension/untension pull wires/tendons 45 of the scope 40 to cause articulation of the scope 40.

The scope 40 can further include one or more working channels 44, which may be formed inside the elongate shaft and run a length of the scope 40. The working channel 44 may serve for deploying therein a medical tool 35 or a component of the medical instrument 32 (e.g., a lithotripter, a basket, forceps, laser, or the like) or for performing irrigation and/or aspiration, out through a distal end of the scope 40, into an operative region surrounding the distal end. The medical instrument 32 may be used in conjunction with a medical tool 35 and include various hardware and control components for the medical tool 35 and, in some instances, include the medical tool 35 as part of the medical instrument 32. For example, as shown, the medical instrument 32 can comprise a basket formed of one or more wire tines but any medical tool 35 are contemplated.

### Mapping, Navigation, and Positioning Modalities/Systems

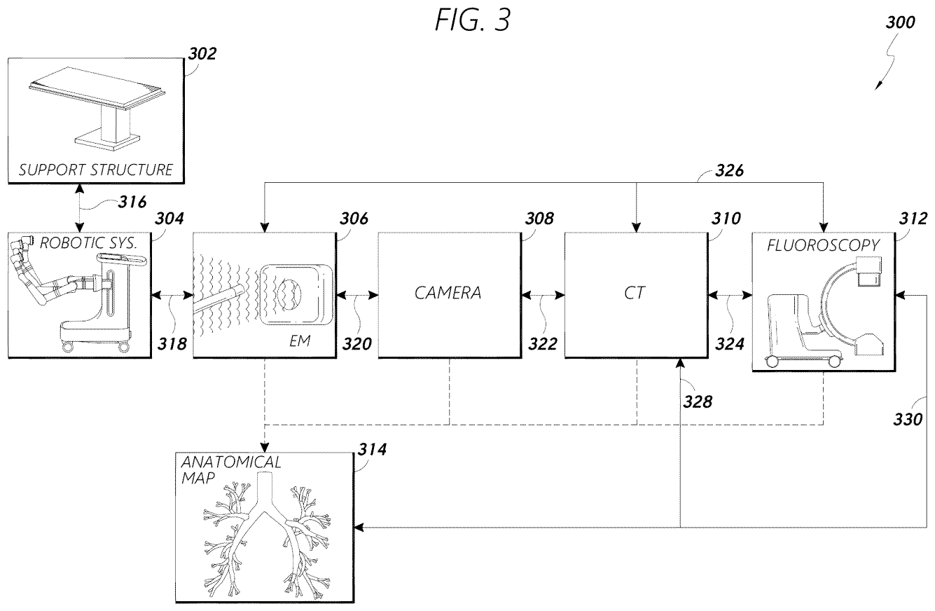

FIG. 3 is a block diagram illustrating a system 300 including various positioning and/or imaging systems/modalities 302-312 (sometimes referred to as “subsystems”), which can be implemented to facilitate anatomical mapping, navigation, positioning, and/or visualization for procedures in accordance with one or more examples. For example, the various systems 302-312 can be configured to provide data for generating an anatomical map, determining a location of an instrument, determining a location of a target, and/or performing other techniques.

Each of the systems 302-312 can be associated with a respective coordinate frame (also referred to as “position coordinate frame’) and/or can provide data/information relating to instrument and/or anatomy locations, wherein registering the various coordinate frames to one another can allow for integration of the various systems to provide mapping, navigation, and/or instrument visualization. For example, registration of various modalities to one another can allow for determined positions in one modality to be tracked and/or superimposed on/in a reference frame associated with another modality, thereby providing layers of positional information that can be combined to provide a robust localization system.

In examples, the system 300 is configured to implement one or more localization/localizing techniques (also referred to as “localization/localizing system 300”). Localization/localizing can refer to processes of determining a location and orientation/pose of an instrument or other element/component within a given space or environment.

In various examples, the anatomical space in which a medical instrument can be localized (i.e., where position and/or shape of the instrument is determined/estimated) is a 2D or 3D portion of a subject's tracheobronchial airways, vasculature, urinary tract, gastrointestinal tract, or any organ or space accessed via lumens. Various modalities can be implemented to provide images/representations/models of the anatomical space using various imaging techniques described in relation to the imaging system 122 of FIG. 1. One or both of preoperative and intraoperative images can be acquired in connection with a procedure.

The systems 302-312 can provide information for generating a 2D or 3D anatomical model/map 314 (e.g., airway model). In examples, the anatomical map 314 and/or other localization information can be displayed to a user, such as the operating user 5, during a procedure to assist the user in perform the procedure. For example, a visualization of a tracked instrument can be superimposed on the anatomical map 314 based on position/sensor data associated with the tracked medical instrument.

As shown, the system 300 can include a surgical bed or other subject platform or positioning/support structure 302 (e.g., the table 15 of FIG. 1). The position of the support structure 302 can be known based on data maintained relating to the position of the support structure 302 within the surgical/procedure environment. Alternatively, or additionally, the position of the support structure 302 can be sensed or otherwise determined using one or more markers and/or an appropriate imaging/positioning modality.

The system 300 can further include a robotic system 304, such as the robotic system 10 (e.g., a robotic cart or other device or system including one or more robotic end effectors). Data relating to the position and/or state of robotic arms, actuators, and/or other components of the robotic system 304 can be known or derived from robotic command data or other robotic data relative to a coordinate frame of the robotic system 304. In some examples, reference frame registration 316 occurs between the support structure 302 and the robotic system 304, which can be a relatively coarse registration (in some cases) based on robotic system/cart-set-up procedure (which can have any suitable or desirable scheme).

The system 300 can further include an electromagnetic (EM) sensor system 306, which can include an EM field generator (e.g., the EM field generator 120) and one or more EM sensors. An EM sensor can be associated with a portion of an instrument that is tracked/controlled, such as along a length of the instrument and/or other elongate member disposed in the working channel of the instrument. In some implementations, the EM field generator can be mechanically coupled to either the support structure 302 or the robotic system 304, in which case registration/association 318 between such systems can be known and/or determined. In some implementations, the registration 318 between the EM sensor system 306 and the robotic system 304 can be determined through forward kinematics and/or field generator mount transform information. For example, the field generator can be mounted to an end effector/manipulator of the robotic system 304, such that the position of the field generator can be known relative to the robotic system positioning frame based on the known relationship between the position of the robotic end effector and the robotic system 304. The EM sensor system 306 can provide instrument pose and/or path information based on sensor readings associated with the instrument.

The system 300 can further include an optical camera system 308 including one or more cameras or other imaging devices, wherein such device(s) is/are configured to generate images of subject anatomy within a visual field thereof, such as real-time image data during a surgical procedure. In examples, registration 320 between the optical camera system 308 and the EM sensor system 306 can be achieved through identification of features having EM sensor data associated therewith, such as by a medical instrument tip, in images generated by the optical camera system 308. The registration 320 can further be based at least in part on hand-eye interaction of the physician when viewing real-time camera images while the EM-sensor-equipped endoscope is navigating in the subject anatomy.

The system 300 can further include a computed tomography (CT) imaging system 310 configured to generate CT images of the subject anatomy, which can be done preoperatively and/or intraoperatively. Specifically, CBCT is a type of CT imaging technique that uses a cone-shaped X-ray beam and a specialized detector to capture multiple two-dimensional (2D) X-ray images from different angles around the patient. In CBCT imaging, a 3D volumetric representation can be reconstructed using the 2D images.

In examples, image processing can be implemented for registration 322 of the CT image data with the camera image data generated by the optical camera system 308. For example, common features identified in both camera image data and CT image data can be identified to relate the CT image frame to the camera image frame in space. In some examples, the CT imaging system 310 can be used to generate preoperative imaging data for producing the anatomical map 314 and/or for path navigation planning.

In examples, the fluoroscopy imaging system 312 can be registered 324 to the CT imaging system 310 using any image processing technique suitable for such registration. Fluoroscopy can provide real-time, continuous X-ray imaging of moving structures inside the body. The images produced during fluoroscopy are typically two-dimensional and show a dynamic view of the area being examined. This real-time imaging can enable the operator 5 to visualize the movement and position of anatomical structures or medical instruments 32 during procedures. Although the CT imaging system 310 and fluoroscopy imaging system 312 are illustrated as separated systems, in examples the same system may perform the functionality of the CT imaging system 310 and fluoroscopy imaging system 312.

In examples, the CT imaging system 310 and/or the fluoroscopy imaging system 312 can be registered 326 to the EM sensor system 306 through various techniques, such as tool registration, a transformation function, etc. In one example, a mechanical structure of the C-arm instrumentation of the system 310, 312 can have a known physical transform/relationship with respect to a mounting position of the EM field generator of the EM sensor system 306. Such known relationship can be used to register the CT/fluoroscopy image space to the EM sensor image space. The connections 328, 330 represent registrations/relationships of the CT imaging system 310 and the fluoroscopy imaging system 312 to the anatomical map 314, respectively.

The position, shape, and/or orientation of an instrument, such as an endoscope, can be determined using any one or more of the systems 302-312, which can facilitate generation of graphical interface data representing the estimated position and/or shape of the instrument relative to the anatomical map 314. The position, shape, and/or orientation of the instrument and/or anatomical map 314 can be displayed on a display device, such as via the control system 50 and/or robotic system 10, or another device. In some examples, the anatomical map 314 also indicates a position(s) of a target(s), such as a location within the anatomy that has been designated for further treatment.

Although the systems 302-312 are discussed in a specific order, the systems can be implemented in different orders. Moreover, the systems can be used in different ways. Further, registration can occur between different systems.

In some illustrations, one or more of the systems 302-312 can be implemented to generate the anatomical map 314 preoperatively and/or determine a location of one or more targets within the anatomical map 314. Intraoperatively, one or more of the systems 302-312 can also be implemented to determine a location of a medical instrument and/or position of a target relative to the anatomical map 314. As discussed herein, one or more of the systems 302-312 can also be implemented to update the anatomical map 314, location of the medical instrument, location of the target, etc.

### Intraoperative Tip Pose Estimation Using External Imaging Modality

Referring back to the respiratory system example of FIG. 1, it was described that the imaging system 122 including an external imaging modality, such as CBCT, may generate a 3D model (e.g., the anatomical map 314) of the lungs 4. The anatomical map 314 can correspond to a 3D model of a subject's anatomy. Traditionally, the use of external imaging has mostly been limited to the generation of the 3D model for use in preoperative planning and/or as an intraoperative reference model in connection with EM-based or camera-based tip pose estimation.

The EM-based or camera-based tip pose estimation using the anatomical map 314 as a reference model may not be accurate. For example, there may be discrepancies between the anatomical map 314 generated based on preoperatively captured images and actual anatomy of the subject 7 during a medical procedure. As another example, the anatomical map 314 may not capture movement of objects and/or anatomy, gastrointestinal tract, blood vessels, or the scope 40. In some cases, respiration of the subject 7 may cause lung 4 to deform its shape to the extent that the tip may be displaced in the anatomical map 314 at an incorrect location (e.g., a secondary bronchus 78 instead of a tertiary bronchus 75 or in a wrong bronchus).

The present disclosure provides tip pose estimation using images captured during the procedure. CT imaging techniques including CBCT and fluoroscopy can capture or otherwise provide images intraoperatively. These imaging techniques can effectively capture both the tip of the scope (also referred herein as “a tip portion of a scope” or “a scope tip”) and its surrounding anatomical environment, thereby providing valuable insight of the relationship between them. Thus, using the CT images for tip pose estimation and/or localization can help resolve discrepancies between the anatomical map 314 and the actual anatomy by providing an additional reference.

In particular, the present disclosure provides for streamlined CT-based tip pose estimation approaches that are time and resource efficient. The present disclosure contemplates expanding use of the CT-based systems to intraoperative tip pose estimation. The CT-based tip pose estimation can aid the operator 5 in adjusting tip pose when aligning the tip to a target within a 3D space. For example, the CT-based tip pose estimation can help align a scope tip and a biopsy needle carried within a working channel of the scope tip to a nodule (e.g., a nodule 89 in FIG. 1) during biopsy. As another example, the CT-based tip pose estimation can help align a basket within the working channel to a kidney stone during ureteroscopy. The CT-based tip pose estimation is described with greater detail below.

### Tip Pose Estimation Pipeline

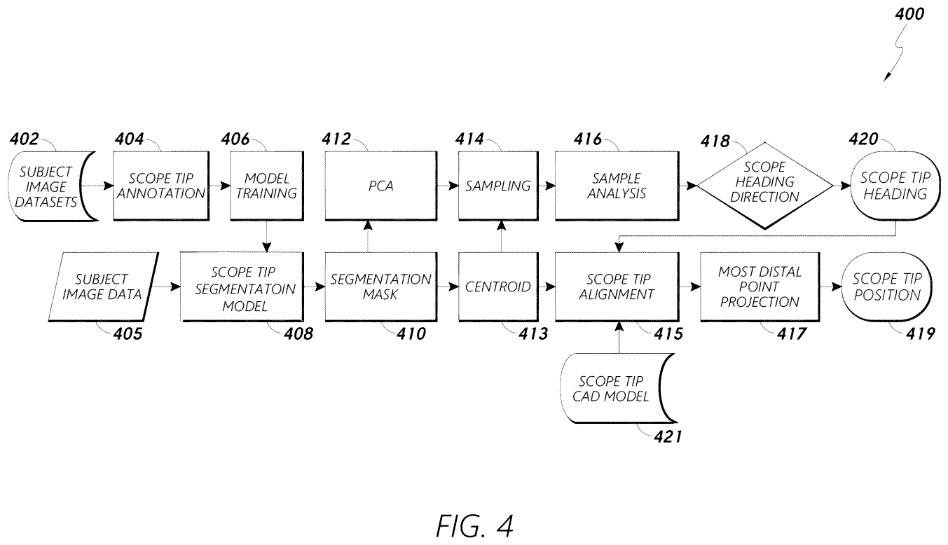

FIG. 4 illustrates an example block diagram of a tip pose estimation pipeline 400, in accordance with one or more embodiments. The pipeline 400 of FIG. 4 visualizes stages or sequences of stages involved in the tip pose estimation process. In particular, the pipeline 400 focuses on data flow and data processing aspects.

At high level, the pipeline 400 can involve accessing externally captured image data, segmenting a scope tip from the image data, generating a volumetric representation of the scope tip, and analyzing the volumetric representation to determine a tip pose (position and/or orientation). In some implementations, segmenting the scope tip can involve use of a machine learning model, such as a deep learning model including one or more neural networks, configured to identify/segment the scope tip from the scope and its surroundings. In some implementations, generating the volumetric representation can involve generating a point cloud and analyzing the volumetric representation can involve determining a principal axis based on principal component analysis (“PCA”) and/or calculating a centroid of the point cloud. Tip pose can be estimated based on the principal axis and the centroid. It will be understood that, while the present disclosure will be described based on specific implementation techniques mentioned above, use of other techniques and many variations are possible.

“Scope tip segmentation model 408” will be understood to refer to the machine learning model. Example suitable machine learning architectures include convolutional neural networks (CNNs), recurrent neural networks (RNNs), long short-term memory (LSTM) networks, and transformer-based architectures (such as vision transformers), among other examples. In some embodiments, the scope tip segmentation model 408 can have a U-Net architecture. It will be understood that the U-Net architecture is one example architecture for the scope tip segmentation model 408 and any type or combination of machine learning model(s) can be used depending on task requirements, available computer resources, and available data. The scope tip segmentation model 408 can be preoperatively trained and intraoperatively used for segmentation of the scope tip.

### I. Training Scope Segmentation Model

During training, the scope tip segmentation model 408 learns to identify/segment a region corresponding to a scope tip. The training can involve preparing the subject image datasets 402, annotating the scope tip in the subject image dataset 402, and training the scope tip segmentation model 408 based on the subject image datasets 402.

During training, the pipeline 400 can involve accessing (e.g., receiving, retrieving, capturing, decompressing, etc.) original images depicting a scope, a scope tip thereof, and anatomy surrounding the scope (e.g., tissues, bones, etc.). The pipeline 400 can also involve preparing subject image datasets 402 based on the original images. The original images may be externally captured images (e.g., CT images captured by the imaging system 122 positioned external to the subject 7 in FIG. 1) or simulated images. In some embodiments, the original images may be resized, normalized in some visual property (e.g., brightness, gamma correction, etc.), converted into a common format, or the like. In some embodiments, the original images may be altered for increased diversity through transformations (e.g., rotation, scaling, flipping, cropping, clastic deformations, etc.).

In some embodiments, the original images may be CT images, including CBCT images. In CT, an X-ray beam and a detector can capture multiple 2D X-ray images from different angles around a scanned area of the subject 7. A 3D volumetric representation or model of the scanned area may be reconstructed based on the multiple 2D X-ray images. Thus, when referring to CT, it is possible that the multiple 2D X-ray images may collectively form an image dataset and the subject image datasets 402 may include multiple such CT image datasets. Herein, the 2D images or the 3D representation may be referred to as the subject image dataset 402 depending on context.

At scope tip annotation block 404, the pipeline 400 can involve annotating the scope tip in the subject image datasets 402. The annotation involves creating, for each image, a corresponding ground truth segmentation mask. The ground truth segmentation masks are used during training as ground truth labels and are not to be confused with inference segmentation masks generated during inference. In a ground truth segmentation mask, each pixel in the mask is assigned a label or a class corresponding to the object or a background label if it does not belong to any object.

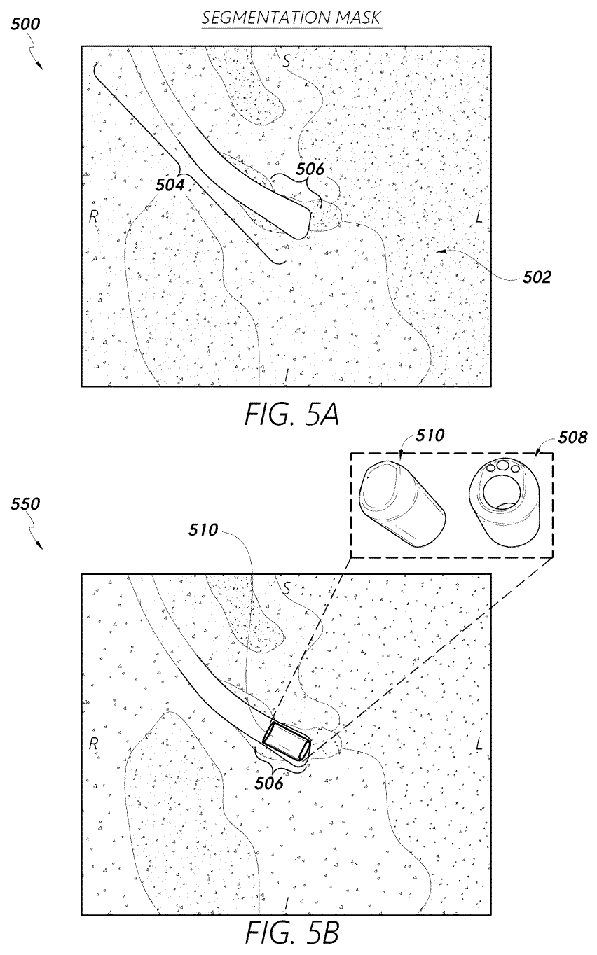

FIG. 5A illustrates an example original image 500 depicting a scope 504 and its scope tip 506 both of which are surrounded by tissue 502. The example original image 500 may be a 2D slice of a CT image dataset collectively associated with a 3D reconstruction. Accordingly, in some embodiments, annotation of the ground truth segmentation mask may involve assigning a label or a class to voxels of a 3D volumetric representation reconstructed from the CT image dataset, thus creating a 3D segmentation mask or multiple 2D slices of segmentation masks.

Although some embodiments of the annotation block 404 may involve manual identification of pixels/voxels in segmentation masks, the present disclosure additionally contemplates semi-automated annotation using a known geometry (e.g., physical dimensions and shape) of the scope tip. The known geometry may be supplied by a known 3D model, which may be a computer-aided design (CAD) model of the scope tip. FIG. 5B illustrates an example annotated image 550 depicting identification of pixels/voxels in the example original image 500 using a CAD model 508 (or a simpler model generated from the CAD model, such as a convex hull model 510). The convex hull model 510, being the smallest convex shape or polygon that encloses all points in the CAD model 508, is without any concavities and may simplify annotation/creation of segmentation masks. While the CAD model 508 and the convex hull model 510 are described as example 3D models, other 3D models including bounding boxes, bounding spheres, alpha shapes, or the like are contemplated.

As shown in the example annotated image 550, the convex hull model 510 can be positioned and oriented to align on the scope tip 506. For example, the convex hull model 510 can be placed to align with the scope tip 506 in 2D CT image slices or a 3D reconstruction generated based on the 2D CT image slices. In some embodiments, the convex hull model 510 can be converted to a segmentation node configured to generate the ground truth segmentation masks of the scope tip. The ground truth segmentation masks can be provided to model training block 406.

At model training block 406, the scope tip segmentation model 408 (e.g., a neural network model) can be trained using the subject image datasets 402 and corresponding ground truth segmentation masks. For example, weights in the neural network model can be adjusted to minimize error during supervised learning. The subject image datasets 402 may be split into training datasets and validation datasets. The trained scope segmentation model 408 may be evaluated for segmentation task accuracy using various metrics, including the Dice similarity coefficient configured to measure similarity between segmentation masks and ground truth labels.

It will be understood that the training of the scope tip segmentation model 408 may be performed offline and/or on a specialized hardware (e.g., server cloud) while inference using the scope tip segmentation model 408 may be performed online and in connection with the medical system 100. Furthermore, it will be understood that the scope tip segmentation model 408 used in the training can be modified in some aspects before deployment. For example, any of quantization, pruning, model compression, or other optimizations may ready the trained scope tip segmentation model 408 for deployment.

### II. Inference Using Scope Segmentation Model

During inference, at segmentation mask block 410, the pipeline 400 can involve generating segmentations masks for new, yet unseen image data using the trained scope tip segmentation model 408. The new image data can be subject image data 405 captured intraoperatively or near real-time and provided as input to the scope tip segmentation model 408. The subject image data 405 can be a CT image dataset including 2D slices (or a 3D reconstruction based on the 2D slices) depicting a scope tip. As output from the scope tip segmentation model 408, 2D/3D segmentation masks corresponding to the CT images can be generated.

The segmentation masks can be a map labelling each pixel/voxel as a scope tip or otherwise. In some implementations, the map can be a binary image that represents pixels/voxels corresponding to the scope tip with a first value (e.g., “1”) and background with a second value (e.g., “0”). The generated segmentation masks can identify/segment a region corresponding to the scope tip in the subject image data 405.

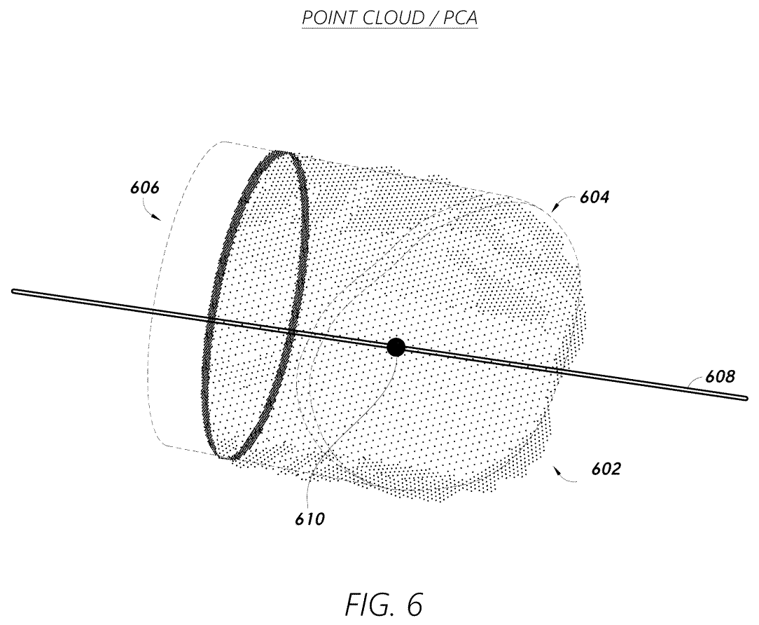

A point cloud representing the scope tip in 3D space can be generated based on the 2D/3D segmentation masks generated at block 410. In some implementations, the point cloud can be a volume-fused set of 2D segmentation masks or the 3D segmentation mask itself. FIG. 6 illustrates an example point cloud 602, in accordance with one or more embodiments. In some implementations, the point cloud 602 can be a collection of all pixels/voxels labeled as the scope tip. In the example of FIG. 6, a silhouette 606 of a 3D model 604 (e.g., the convex hull model 510 of FIG. 5B) of the tip is overlaid on top of the point cloud 602 to provide a sense of which points of the point cloud 602 correspond to which portion of the tip.

At principal component analysis (PCA) block 412, the pipeline 400 can involve performing a principal component analysis (PCA) to determine one or more principal axes (also known as “principal components”) where a point cloud has the most variance. PCA as applied to a point cloud can find principal axes that capture the directions of highest variance in the point cloud. Of the three principal axes possible for a point cloud in 3D space, a principal axis associated with the maximum variance can be selected. Referring to the point cloud 602, performing PCA can find a principal axis 608 which provides the maximum variance in the point cloud. The principal axis 608 is an axis that extends indefinitely and without a directional sense in relation to a reference point.

At centroid block 413, the pipeline 400 can involve determining a centroid (akin to a center of mass) for a point cloud. As an example, the centroid 610 can be found for the point cloud 602. Based on the centroid 610 as a reference point, the principal axis 608 can be split into two possible directions as shown, each direction facing away from the centroid 610. Either of the two possible directions can correspond to a tip direction (e.g., directional components of a 3D vector). In the present disclosure, the tip direction may be referred to as the “scope heading direction” or “scope tip heading,” either of which are reflective of tip orientation.

The pipeline 400 can estimate scope tip pose, including scope tip heading 420 and scope tip position 419, based on the principal axis and the centroid.

### III. Scope Tip Heading Determination

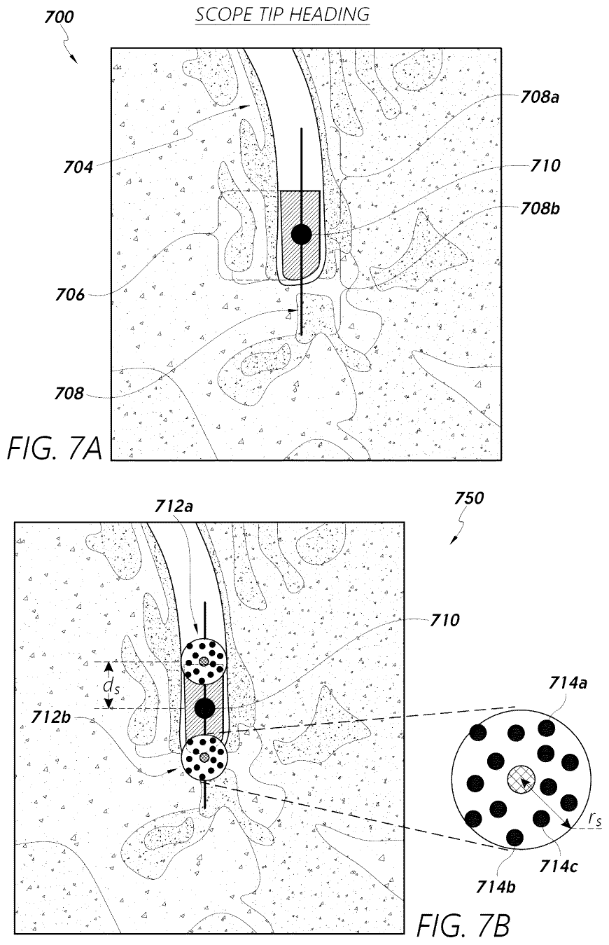

As described, two possible scope tip heading facing away from a centroid may be identified on a principal axis having the maximum variance. FIG. 7A illustrates an example CT image 700 depicting a scope 704 and a scope tip 706. It will be understood that the example CT image 700 is a cross-sectional image of a 3D representation of the scope tip 706 (e.g., an image in the subject image data 405 of FIG. 4). The example CT image 700 shows a centroid 710 and a first direction 708a facing away from the scope tip toward the back of the scope tip and a second direction 708b that orients the scope toward the tip. Selecting the correct scope tip heading between the first direction 708a or the second direction 708b can involve sampling pixel/voxel values and performing sample analysis based on the sampled values.

At sampling block 414, the pipeline 400 can involve creating two diametrically opposed sampling regions, 2D or 3D, and sampling points therein. For example, FIG. 7B illustrates an example sampling image 750 with two sampling regions configured as diametrically opposed spheres 712a, 712b along the principal axis 708. A first sphere 712a is positioned at the proximal end of the scope tip and a second sphere 712b is positioned at the distal end of the scope tip. The spheres 712a, 712b may be of the same size or different sizes.

The spheres 712a, 712b can be positioned on the principal axis 708 based on a known geometry of the scope tip. For example, centers of the spheres 712a, 712b can be separated by a separation distance (denoted dₛ) that is approximately half of the length of a scope tip in a CAD model (e.g., the CAD model 508 of FIG. 5B) of the scope tip. Similarly, the spheres 712a, 712b can be set to have a sampling radius (denoted rₛ) that matches, exactly or approximately, with a radius of the cylindrical portion in the CAD model. As examples, the separation distance can be approximately 6 millimeters and the sampling radius approximately 3 millimeters.

Within the sampling regions, pixel/voxel values can be sampled at sampling points 714. In some implementations, the sampling points 714 may be evenly distributed or distributed based on a known profile. It will be understood that, while spheres 712a, 712b are used as example sampling regions, any regions in 2D or 3D can be used including, for example, circles, triangles, rectangles, cubes, cones, cylinders, pyramids, ellipsoids, or the like. Additionally, it will be noted that the separation distance (dₛ), the sampling radius (rₛ), a number of sampling points 714, a distribution profile of the sampling points 714, or the like are configuration values and may be tuned based on CT images, scope tip, or both.

In CT images, the pixel/voxel value associated with each sampling point 714a, 714b, 714c can represent a degree of radiodensity in the subject image data 405. Hounsfield unit (HU) is a measurement scale used to quantify radiodensity of tissues and materials. The Hounsfield unit scale assigns numerical values to different tissues and materials based on their X-ray attenuation properties, where metallic objects and dense structures that absorb more X-rays have higher HU unit values (more attenuation) compared to soft tissues and fluids that absorb less X-rays (lower attenuation). Denser materials associated with higher HU unit values appear brighter (e.g., with greater brightness intensity) than sparse materials associated with lower HU unit values, which appear darker (e.g., with lesser brightness intensity).

At sample analysis block 416, the pipeline 400 can involve analyzing pixel/voxel values sampled at the sampling points 714. In the example sampling image 750, all or substantially all sampling points 714 of the first sphere 712a are pixels/voxels depicting the scope. In other words, most of the first sphere 712a overlaps the scope. In contrast, only about half of the sampling points of the second sphere 712b are pixels/voxels depicting the scope while the other half of the sampling points of the second sphere 712b are pixels/voxels that do not depict the scope (but rather, depict objects or features surrounding the scope, such as tissue, bone, air, or other anatomical features). In other words, only half of the second sphere 712b overlaps the scope. Thus, the sum of pixel/voxel values within the first sphere 712a is greater (e.g., a higher sum of sampled HU values) than the sum of pixel/voxel values within the second sphere 712b (e.g., a lower sum of sampled HU values).

At scope heading direction block 418, the correct scope tip heading direction can be selected based on a result of the sample analysis block 416. For example, the second direction 708b in FIG. 7A having the lower sum may be selected as the correct scope tip heading direction based on a comparison of the pixel/voxel values.

In some embodiments, only one region (e.g., either of the first sphere 712a or the second sphere 712b, but not both) may be sampled for pixel/voxel values within the region and compared against a threshold level. In other words, a single vector (pointing from the centroid 710 to the sampling region) can be tested for the scope tip direction. For example, a threshold level may be set as a value between the first sum and the second sum so that, when a sum of the pixel/voxel values within the region is greater than the threshold level, a direction opposite the vector may be selected as the scope tip direction. In other words, the scope tip direction will be a negated version of the vector. On the other hand, when the sum is less than the threshold level, the direction of the vector may be selected as the scope tip direction.

The scope tip direction, as determined by the scope heading direction block 418, is the scope tip heading 420. In the example CT image 700, the scope tip heading 420 is the second direction 708b. In some implementations, the scope tip heading 420 may be represented in 3D space as a vector.

### IV. Scope Tip Position Determination

The scope tip position may be determined based on a centroid, a scope tip CAD model 421, and a scope tip heading 420.

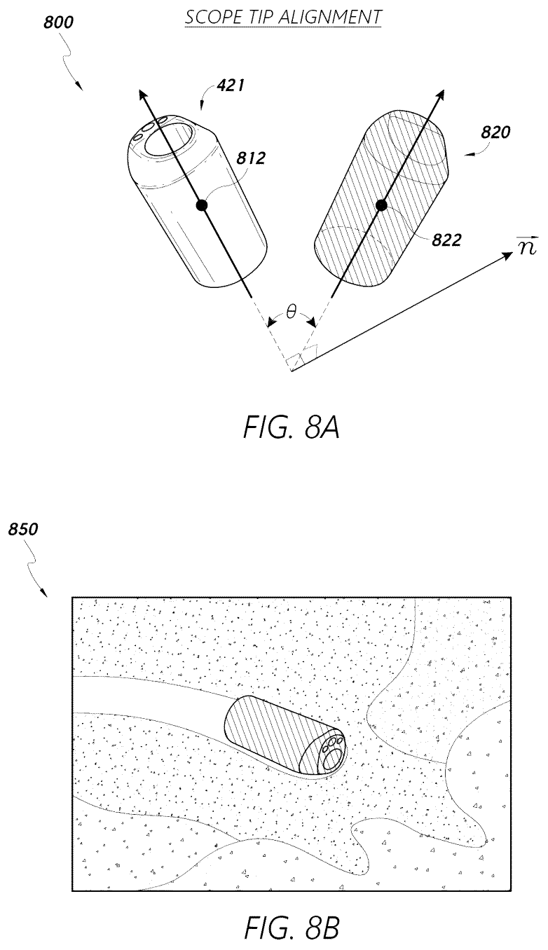

At scope tip alignment block 415, the pipeline 400 can involve mapping/registering and/or aligning the scope tip CAD model 421 to a 3D reconstructed tip (e.g., a point cloud or another volumetric representation). FIG. 8A illustrates an example alignment process 800 depicting the scope tip CAD model 421 having a first centroid 812 on a first principal axis (e.g., a Z-axis of a CAD coordinate frame) and a 3D reconstructed tip 820 having a second centroid 822 on a second principal axis (e.g., the scope tip heading 420). The 3D reconstructed tip 820 may be a convex hull model reconstructed based on segmentation masks inferred by the scope tip segmentation model 408.

The alignment can involve determining a transform that aligns the first principal axis with the second principal axis and mapping the first centroid 812 to the second centroid 822. In vector space, the alignment can involve a rotation (denoted θ) of the scope tip CAD model 421 onto the 3D reconstructed tip 820 such that the CAD model 421 is rotated about a normal vector (denoted n) onto the 3D reconstructed tip 820. In some embodiments, the transform may additionally “roll” (not shown) the scope tip CAD model 421 during the alignment to account for the roll of the 3D reconstructed tip. FIG. 8B illustrates a successful alignment 850 where a CAD model is overlaid on a 3D reconstructed tip.

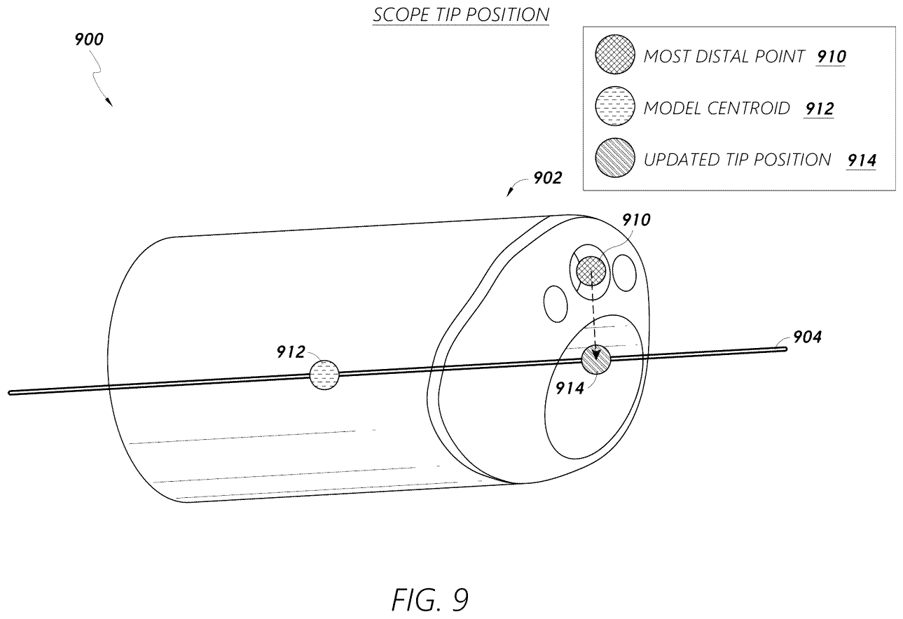

At most distal point projection block 417, the pipeline 400 can involve projecting the most distal point in a 3D model onto a principal axis having the maximum variance. For example, FIG. 9 illustrates an example rendering 900 depicting a projection of the most distal point 910 of a scope tip CAD model 902 (e.g., the scope tip CAD model 421) onto a principal axis 904 positioned through a model centroid 912. The scope tip CAD model 902 has a known shape and, thus, the most distal point of the 3D model (relative to the centroid 912) is known. The projection of the most distal point 910 onto the principal axis 904 can represent an updated tip position 914 on the principal axis 904 (such as the scope tip position 419).

The scope tip heading 420 can represent tip orientation and the scope tip position 419 represent the tip position. Accordingly, the pipeline 400 can estimate the tip pose using externally captured images. It will be understood that depicted blocks in the pipeline 400 are exemplary and that fewer or more blocks, as well as blocks organized in different orders, may be involved in the tip pose estimation. Furthermore, it will be understood that depicted blocks may be carried out in parts and/or on separate systems. For example, the scope tip annotation block 404 and the model training block 406 may be carried out in their entirety during the training step on a cloud server while other blocks are carried out at a much later time on the medical system 100. Many other variations are also possible.

### Scope Tip Pose Estimation Workflow

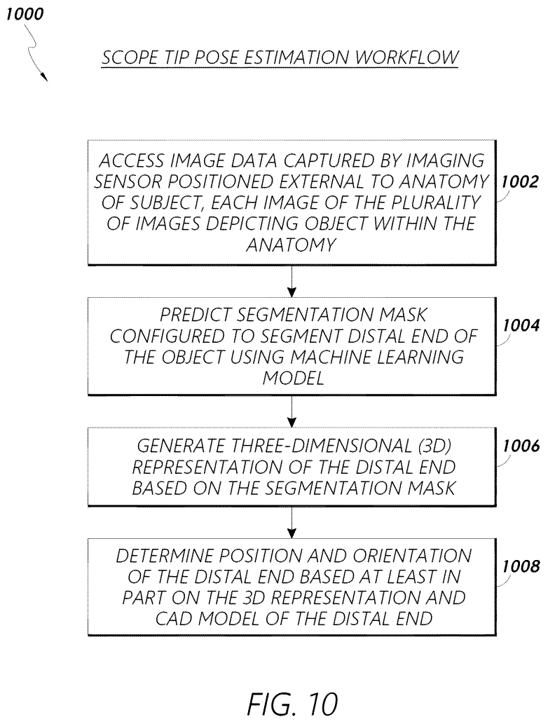

FIG. 10 illustrates a flow diagram illustrating an instrument tip pose estimation process 1000, in accordance with one or more embodiments. The process 1000, when followed, can determine scope tip heading and scope tip position from externally captured images.

At block 1002, the process 1000 can involve accessing image data of a plurality of images captured by an imaging system positioned external to an anatomy of a subject. The images can be CT images including CBCT images. Each image of the plurality of images can depict an object within the anatomy.

At block 1004, the process 1000 can involve predicting at least one segmentation mask configured to segment a distal end of the object using a machine learning model. The object can be a scope and the distal end can be a tip of the scope. In some embodiments, the machine learning model may be trained using ground truth segmentation masks annotated with a 3D model (e.g., the convex hull model 510 of FIG. 5B of the scope tip) of the distal end.

At block 1006, the process 1000 can involve generating a 3D representation of the distal end based on the at least one segmentation mask. The segmentation masks can be used to reconstruct the 3D representation of the distal end. In some embodiments, the 3D representation may be a point cloud.

At block 1008, the process 1000 can involve determining either a position or an orientation or both of the distal end based at least in part on the 3D representation and a CAD model of the distal end. The orientation of the distal end can be determined based on aligning the respective centroids and the principal axes of the 3D representation and the CAD model. In some embodiments, points along the aligned principal axes may be sampled for comparison to eliminate candidate (but incorrect) orientations to identify a correct direction of the orientation. The position may be determined by projecting the most distal point of the CAD model onto the aligned principal axes.

It will be understood that depicted blocks in the process 1000 are exemplary and fewer or more blocks, as well as blocks organized in different orders, may be involved in the tip pose estimation. Many variations are possible.

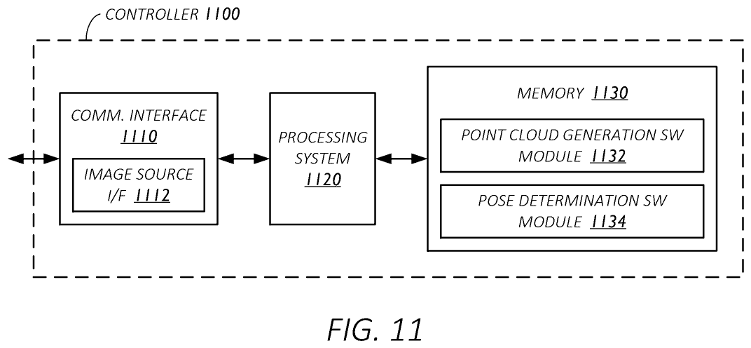

FIG. 11 shows another block diagram of an example controller 1100 for a medical system, according to some implementations. In some implementations, the controller 1100 may be one example of the control circuitry 251 and/or 211 of FIG. 2. More specifically, the controller 1100 is configured to estimate the pose of an object within an anatomy. With reference for example to FIG. 4, the controller 110 may implement one or more stages of the tip pose estimation pipeline 400.

The controller 1100 includes a communication interface 1110, a processing system 1120, and a memory 1130. The communication interface 1110 is configured to communicate with one or more components of the medical system. More specifically, the communication interface 1110 includes an image source interface (I/F) 1112 for communicating with one or more image sources (such as the CT imaging system 310 and/or the fluoroscopy imaging system 312 of FIG. 3). In some implementations, the image source I/F 1112 may receive image data representing a 3D model of an anatomy having an instrument disposed therein.

The memory 1130 may include a non-transitory computer-readable medium (including one or more nonvolatile memory elements, such as EPROM, EEPROM, Flash memory, or a hard drive, among other examples) that may store the following software (SW) modules: a point cloud generation SW module 1132 to generate a point cloud associated with a distal end of the instrument based on the image data; and a pose determination SW module 1134 to determine a pose of the distal end of the instrument based at least in part on the point cloud and a known geometry of the distal end of the instrument.

The processing system 1120 may include any suitable one or more processors capable of executing scripts or instructions of one or more software programs stored in the controller 1100 (such as in the memory 1130). For example, the processing system 1120 may execute the point cloud generation SW module 1132 to generate a point cloud associated with a distal end of the instrument based on the image data. The processing system 1120 may further execute the pose determination SW module 1134 to determine a pose of the distal end of the instrument based at least in part on the point cloud and a known geometry of the distal end of the instrument.

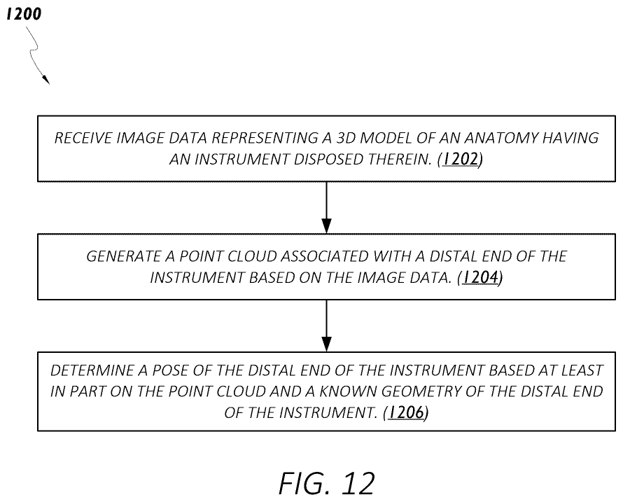

FIG. 12 shows an illustrative flowchart depicting an example pose estimation operation 1200, according to some implementations. In some implementations, the example operation 1200 may be performed by a controller for a medical system such as the controller 1100 of FIG. 11.

The controller receives image data representing a 3D model of an anatomy having an instrument disposed therein (1302). The controller generates a point cloud associated with a distal end of the instrument based on the image data (1304). The controller further determines a pose of the distal end of the instrument based at least in part on the point cloud and a known geometry of the distal end of the instrument (1306).

In some aspects, the controller may further segment the distal end of the instrument from the 3D model based on a machine learning model trained to infer a segmentation mask from the image data, where the point cloud is generated based on the segmentation mask. In some implementations, the machine learning model may be trained based at least in part on a 3D model of the distal end of the instrument that is generated based on the known geometry. In some implementations, the 3D model may be a convex hull model.

In some aspects, the determining of the pose of the distal end of the instrument may include determining a principal axis that maximizes a variance of the point cloud; selecting, on the 3D model, a first sampling region aligned with the principal axis at a predetermined distance in a first direction from a centroid of the point cloud, where the predetermined distance and dimensions of the first sampling region are configured based on the known geometry of the distal end of the instrument; sampling voxel values of the image data within the first sampling region; and determining an orientation of the distal end of the instrument based at least in part on the sampled voxel values within the first sampling region.

In some implementations, the determining of the orientation of the distal end of the instrument may include determining whether a sum of the voxel values within the first sampling region exceeds a threshold value. In some other implementations, the determining of the orientation of the distal end of the instrument may include selecting, on the 3D model, a second sampling region aligned with the principal axis at the predetermined distance in a second direction from the centroid of the point cloud; sampling voxel values of the image data within the second sampling region; and determining whether a sum of the voxel values within the first sampling region is greater than a sum of the voxel values within the second sampling region.

In some other aspects, the determining of the pose of the distal end of the instrument may include mapping a 3D model of the distal end of the instrument to the point cloud, where the 3D model of the distal end is generated based on the known geometry; and determining a position of the distal end of the instrument based at least in part on the mapping of the 3D model of the distal end to the point cloud. In some implementations, the determining of the position of the distal end of the instrument may include determining a principal axis that maximizes a variance of the point cloud; and projecting, onto the principal axis, a point on the 3D model of the distal end of the instrument furthest from a centroid of the 3D model of the distal end, where the projected point represents the position of the distal end of the instrument.

### ADDITIONAL EMBODIMENTS

Depending on the embodiment, certain acts, events, or functions of any of the processes or algorithms described herein can be performed in a different sequence, may be added, merged, or left out altogether. Thus, in certain embodiments, not all described acts or events are necessary for the practice of the processes.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, element, etc. may be either X, Y or Z. Thus, such conjunctive language is not generally intended to imply that certain embodiments require at least one of X, at least one of Y and at least one of Z to each be present.

It should be appreciated that in the above description of embodiments, various features are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular embodiment herein can be applied to or used with any other embodiment(s). Further, no component, feature, step, or group of components, features, or steps are necessary or indispensable for each embodiment. Thus, it is intended that the scope of the inventions herein disclosed and claimed below should not be limited by the particular embodiments described above, but should be determined only by a fair reading of the claims that follow.

It should be understood that certain ordinal terms (e.g., “first” or “second”) may be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather may generally distinguish the element from another element having a similar or identical name (but for use of the ordinal term). In addition, as used herein, indefinite articles (“a” and “an”) may indicate “one or more” rather than “one.” Further, an operation performed “based on” a condition or event may also be performed based on one or more other conditions or events not explicitly recited.

Unless otherwise defined, all terms (including technical and scientific terms) used herein have the same meaning as commonly understood by one of ordinary skill in the art to which example embodiments belong. It is further understood that terms, such as those defined in commonly used dictionaries, should be interpreted as having a meaning that is consistent with their meaning in the context of the relevant art and not be interpreted in an idealized or overly formal sense unless expressly so defined herein. As used herein, the term “patient” may generally refer to humans, anatomical models, simulators, cadavers, and other living or non-living objects.

The spatially relative terms “outer,” “inner,” “upper,” “lower,” “below,” “above,” “vertical,” “horizontal,” and similar terms, may be used herein for ease of description to describe the relations between one element or component and another element or component as illustrated in the drawings. It be understood that the spatially relative terms are intended to encompass different orientations of the device in use or operation, in addition to the orientation depicted in the drawings. For example, in the case where a device shown in the drawing is turned over, the device positioned “below” or “beneath” another device may be placed “above” another device. Accordingly, the illustrative term “below” may include both the lower and upper positions. The device may also be oriented in the other direction, and thus the spatially relative terms may be interpreted differently depending on the orientations.

Unless otherwise expressly stated, comparative and/or quantitative terms, such as “less,” “more,” “greater,” and the like, are intended to encompass the concepts of equality. For example, “less” can mean not only “less” in the strictest mathematical sense, but also, “less than or equal to.”

What is claimed is:

1. A controller for a medical system, comprising:
a processing system; and
a memory storing instructions that, when executed by the processing system, cause the controller to:
receive image data representing a three-dimensional (3D) model of an anatomy having an instrument disposed therein;
generate a point cloud associated with a distal end of the instrument based on the image data; and
determine a pose of the distal end of the instrument based at least in part on the point cloud and a known geometry of the distal end of the instrument.

2. The controller of claim 1, wherein execution of the instructions further causes the controller to:
segment the distal end of the instrument from the 3D model based on a machine learning model trained to infer a segmentation mask from the image data, the point cloud generated based on the segmentation mask.

3. The controller of claim 2, wherein the machine learning model is trained based at least in part on a 3D model of the distal end of the instrument that is generated based on the known geometry.

4. The controller of claim 3, wherein the 3D model of the distal end of the instrument is a convex hull model.

5. The controller of claim 1, wherein the determining of the pose of the distal end of the instrument comprises:
determining a principal axis that maximizes a variance of the point cloud;
selecting, on the 3D model, a first sampling region aligned with the principal axis at a predetermined distance in a first direction from a centroid of the point cloud, the predetermined distance and dimensions of the first sampling region configured based on the known geometry of the distal end of the instrument; and
sampling voxel values of the image data within the first sampling region; and
determining an orientation of the distal end of the instrument based at least in part on the sampled voxel values within the first sampling region.

6. The controller of claim 5, wherein the determining of the orientation of the distal end of the instrument comprises:
determining whether a sum of the voxel values within the first sampling region exceeds a threshold value.

7. The controller of claim 6, wherein the determining of the orientation of the distal end of the instrument further comprises:
determining that the instrument is oriented in the first direction if the sum of the voxel values does not exceed the threshold value; and
determining that the instrument is oriented in a second direction, opposite the first direction, if the sum of the voxel values exceeds the threshold value.

8. The controller of claim 5, wherein the determining of the orientation of the distal end of the instrument comprises:
selecting, on the 3D model, a second sampling region aligned with the principal axis at the predetermined distance in a second direction from the centroid of the point cloud;
sampling voxel values of the image data within the second sampling region; and
determining whether a sum of the voxel values within the first sampling region is greater than a sum of the voxel values within the second sampling region.

9. The controller of claim 8, wherein the determining of the orientation of the distal end of the instrument further comprises:
determining that the instrument is oriented in the first direction if the sum of the voxel values within the first region is not greater than the sum of the voxel values within the second region; and
determining that the instrument is oriented in the second direction if the sum of the voxel values within the first region is greater than the sum of the voxel values within the second region.

10. The controller of claim 1, wherein the determining of the pose of the distal end of the instrument comprises:
mapping a 3D model of the distal end of the instrument to the point cloud, the 3D model of the distal end generated based on the known geometry; and
determining a position of the distal end of the instrument based at least in part on the mapping of the 3D model of the distal end to the point cloud.

11. The controller of claim 10, wherein the determining of the position of the distal end of the instrument comprises:
determining a principal axis that maximizes a variance of the point cloud; and
projecting, onto the principal axis, a point on the 3D model of the distal end of the instrument furthest from a centroid of the 3D model of the distal end, the projected point representing the position of the distal end of the instrument.

12. A method of pose estimation, comprising:
receiving image data representing a three-dimensional (3D) model of an anatomy having an instrument disposed therein;
generating a point cloud associated with a distal end of the instrument based on the image data; and
determining a pose of the distal end of the instrument based at least in part on the point cloud and a known geometry of the distal end of the instrument.

13. The method of claim 12, further comprising:
segmenting the distal end of the instrument from the 3D model based on a machine learning model trained to infer a segmentation mask from the image data, the point cloud generated based on the segmentation mask.

14. The method of claim 13, wherein the machine learning model is trained based at least in part on a 3D model of the distal end of the instrument that is generated based on the known geometry.

15. The method of claim 14, wherein the 3D model of the distal end of the instrument is a convex hull model.

16. The method of claim 12, wherein the determining of the pose of the distal end of the instrument comprises:
determining a principal axis that maximizes a variance of the point cloud;
selecting, on the 3D model, a first sampling region aligned with the principal axis at a predetermined distance in a first direction from a centroid of the point cloud, the predetermined distance and dimensions of the first sampling region configured based on the known geometry of the distal end of the instrument;
sampling voxel values of the image data within the first sampling region; and
determining an orientation of the distal end of the instrument based at least in part on the sampled voxel values within the first sampling region.

17. The method of claim 16, wherein the determining of the orientation of the distal end of the instrument comprises:
determining whether a sum of the voxel values within the first sampling region exceeds a threshold value.

18. The method of claim 16, wherein the determining of the orientation of the distal end of the instrument comprises:
selecting, on the 3D model, a second sampling region aligned with the principal axis at the predetermined distance in a second direction from the centroid of the point cloud;
sampling voxel values of the image data within the second sampling region; and
determining whether a sum of the voxel values within the first sampling region is greater than a sum of the voxel values within the second sampling region.

19. The method of claim 12, wherein the determining of the pose of the distal end of the instrument comprises:
mapping a 3D model of the distal end of the instrument to the point cloud, the 3D model of the distal end generated based on the known geometry; and
determining a position of the distal end of the instrument based at least in part on the mapping of the 3D model of the distal end to the point cloud.

20. The method of claim 19, wherein the determining of the position of the distal end of the instrument comprises:
determining a principal axis that maximizes a variance of the point cloud; and
projecting, onto the principal axis, a point on the 3D model of the distal end of the instrument furthest from a centroid of the 3D model of the distal end, the projected point representing the position of the distal end of the instrument.
